(19) United States
(12) Patent Application Publication
(10) Pub. No.: US 2004/0235007 A1
(43) Pub. Date: Nov. 25, 2004

(54) Method for detecting negatively supercoiled DNA

(75) Inventors: Susumu Hirose, Mishima-shi (JP); Kuniharu Matsumoto, Mishima-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: National Institute of Genetics, Mishima-shi (JP)

(21) Appl. No.: 10/699,852

(22) Filed: Nov. 4, 2003

(30) Foreign Application Priority Data
May 23, 2003 (JP) 2003-146059

(51) Int. Cl. C12Q 1/68; G01N 33/53

(52) U.S. Cl. 435/6; 435/7.5

(57) ABSTRACT

To detect intracellular negatively supercoiled DNA conveniently and efficiently.

A method for detecting negatively supercoiled DNA in cells, characterized by including the steps of incorporating biotinylated psoralen into cells, irradiating the cells with long-wavelength UV rays, causing the cells to react with adivin which has been labeled with a color-developing substance, a fluorescent substance, or a chemiluminescent substance, and measuring developed color, emitted fluorescence, or emitted chemiluminescence of the cells.

### TECHNICAL FIELD

The present invention relates to a convenient, efficient method for detecting negatively supercoiled DNA. The method does not require extraction of DNA from cells.

### BACKGROUND ART

DNA has a double-stranded structure having a helical periodicity of average 10.5 base pairs per turn. When free rotation of double strands in a DNA molecule is constrained, positive or negative supercoils can be formed. Such supercoiled DNA is thought to play important roles in transcription and replication. In particular, negatively supercoiled DNA in eukaryotes have been reported to be accumulated in cells where active transcription takes place and thus found abundantly in cancer cells and other cells with promoted transcriptional activity. Accordingly, detection of supercoiled DNA is considered to be useful for detection of promoted transcriptional activity, in particular, detection of cancer.

Conventional methods for detecting supercoiled DNA make use of psoralen which has ability to be selectively bound to negatively supercoiled DNA. Briefly, psoralen is incorporated into cells, and the cells are irradiated with long-wavelength UV rays, whereby the incorporated psoralen is fixed to DNA. Thereafter, DNA is extracted and excised with restriction enzymes. Psoralen-bound DNA is separated from psoralen-unbound DNA through electrophoresis and is transferred onto a membrane. For detection, Southern hybridization is performed (See Non-Patent Documents 1 to 5).

[Non-Patent Document 1]

Methods Enzymol., 212: 319-335 (1992)

[Non-Patent Document 2]

Methods Enzymol., 212: 242-262 (1992)

[Non-Patent Document 3]

EMBO J. 12: 1067-1075 (1993)

[Non-Patent Document 4]

Proc. Natl. Acad. Sci. USA, 89: 6055-6059 (1992)

[Non-Patent Document 5]

Biochemistry, 36: 3151-3158 (1997)

### PROBLEMS TO BE SOLVED BY THE INVENTION

However, the above-mentioned existing methods for detecting supercoiled DNA involve many steps and are cumbersome. Moreover, detection is possible only after cells are lysed. In addition, since the extracted DNA is excised with restriction enzymes and the resultant fragments are used for hybridization, unless the structure of the employed DNA has been completely elucidated, it is impossible to identify the sites where supercoils can be found, preventing analysis of the supercoil structure.

Accordingly, an object of the present invention is to provide a method for detecting negatively supercoiled DNA in cells more conveniently and more efficiently.

In view of the foregoing, the present inventors have conducted careful studies, and have found that when biotinylated psoralen is incorporated into cells and the resultant cells are irradiated with long-wavelength UV rays and then reacted with adivin which has been labeled with a color-developing substance, a fluorescent substance, or a chemiluminescent substance, followed by direct measurement of the developed color, emitted fluorescence, or emitted chemiluminescence of the cells, intracellular negatively supercoiled DNA can be directly visualized. In addition, they have also found that, quite surprisingly, negatively supercoiled DNA is present in locations where conventional methods failed to confirm its presence.

### SUMMARY OF THE INVENTION

Accordingly, the present invention provides a method for detecting negatively supercoiled DNA in cells, characterized by including the steps of incorporating biotinylated psoralen into cells, irradiating the cells with long-wavelength UV rays, causing the cells to react with adivin which has been labeled with a color-developing substance, a fluorescent substance, or a chemiluminescent substance, and measuring developed color, emitted fluorescence, or emitted chemiluminescence of the cells.

The present invention also provides a method for detecting a cell containing negatively supercoiled DNA, characterized by including the steps of incorporating biotinylated psoralen into cells, irradiating the cells with long-wavelength UV rays, causing the cells to react with adivin which has been labeled with a color-developing substance, a fluorescent substance, or a chemiluminescent substance, and measuring developed color, emitted fluorescence, or emitted chemiluminescence of the cells.

### BRIEF DESCRIPTION OF THE DRAWINGS

FIG. 1 shows genomic organization at 87A7 and restriction enzyme sites used for the Southern analysis. Arrows denote the direction of gene transcription. 87A7 is flanked by specialized chromatin structures, scs and scs'. X, XbaI; E, EcOR1; B, BglII.

FIG. 2 shows results of Southern analysis of hsp70 gene crosslinked with psoralen at 87A7 of salivary gland cells. Lanes 1-7: Heat shocked salivary glands with psoralen treatment (lanes 2-7) or without psoralen treatment (lane 1) were analyzed. The salivary gland samples were irradiated with X-rays before crosslinking (lane 3) or after crosslinking (lane 5). The sample in lane 4 was similarly treated but without X-ray. The sample in lane 6 was treated with α-amanitin before treatment with psoralen, and the sample in lane 7 did not receive α-amanitin treatment.

FIG. 3 shows the effect of DNA nicking and transcription inhibition on crosslinking with psoralen. Lanes 1-7: The conditions same as described in FIG. 2 apply. Lane 8: No heat shock treatment. Lane 9: Heat shock treatment was performed.

FIG. 4 shows visualization of psoralen signals in chromosomes.

(A) Biotinylated psoralen signals were detected with Alexa 488-labeled streptavidin (green). (B) DAPI signals (blue). (C) Merged image. Arrows indicate representative interbands and puffs. In some regions, intense psoralen signals were observed only in the boundary portions of interbands or puffs (shown by arrowheads).

FIG. 5 shows the incidences where psoralen signals disappear upon nicking of DNA or inhibition of transcription.

Before crosslinking with biotinylated psoralen, salivary gland samples were irradiated with X-rays to thereby generate nicks in DNA, resulting in relaxation of supercoils (A to C). Samples treated with α-amanitin for inhibition of transcription (D to F). (A to D) Biotinylated psoralen. (B, E) DAPI. (C, F) Merged images. Representative interbands and puffs are indicated by arrows.

FIG. 6 shows distribution of psoralen signals on chromosomes of heat shocked salivary glands.

(A) Biotinylated psoralen. (B) DAPI. (C) Merged image. Major heat shock puffs at 87A, 87C, and 93D are indicated by arrows. Arrowheads represent interbands with psoralen signals which became undetected in heat shocked samples.

FIG. 7 shows the effect of nicking of DNA or inhibition of transcription on psoralen signals in heat shock puffs.

Heat shocked salivary gland samples were irradiated with X-ray (A to C) or treated with α-amanitin (D to F) before crosslinking with biotinylated psoralen. (A, D) Biotinylated psoralen. (B, E) DAPI. (F, C) Merged images. Arrows indicate heat shock puffs at 87A and 87C.

FIG. 8 shows colocalization of psoralen signals with those of nascent RNA on the salivary gland chromosomes.

(A) Biotinylated psoralen. (B) DAPI. (C) Merged image of A and B. (D) Nascent RNA labeled with Br-UTP. (E) Merged image of (B) and (D). (F) Merged image of (A) and (D). Arrows indicate regions exhibiting strong nascent RNA signals and weak psoralen signals.

FIG. 9 shows colocalization of psoralen and nascent RNA signals in heat shock puffs.

(A) Comparison of psoralen signals and nascent RNA signals from the salivary gland chromosomes obtained from heat shocked larvae. (B and D) Detailed inspection on the heat shock puffs at 87A and 87C. Panel C represents signal intensities of biotinylated psoralen (green), nascent RNA (pink), and DAPI (blue) quantified along the region indicated in (B).

### MODES FOR CARRYING OUT THE INVENTION

Cells which may be employed in the present invention are not particularly limited so long as they are eukaryotic. For example, they may be cells of mammals such as humans, rats, and mice; or may be cells of insects such as Drosophila. Tissues and body fluids from eukaryotic organisms may be used as the cells. In the case where human cells are employed, tissue or cells with which cancer is suspected may be used as a specimen.

Biotinylated psoralens, like psoralen, selectively intercalate between base pairs of negatively supercoiled DNA. Upon exposure to long-wavelength UV light; e.g., 365-nm light, intercalated biotinylated psoralen mediates crosslinking of DNA strands via formation of covalent bonds. Therefore, when biotinylated psoralen that has been fixed to supercoiled DNA by means of irradiation with long-wavelength UV light is reacted with adivin which has been labeled with a color-developing substance, a fluorescent substance, or a chemiluminescent substance in the cells, and the color, fluorescence, or chemiluminescence developed or emitted from the psoralen-bound portion is measured, only the supercoil sites of the DNA in the cells can be selectively detected.

The biotinylated psoralens which may be used in the present invention are not particularly limited, so long as they are biotinylated and endowed with photosensitizing property. For example, there may be employed a compound of the following formula (1):

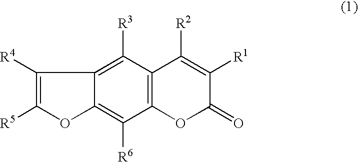

(wherein five members out of R¹ to R⁶ individually represent a hydrogen atom, an alkyl group, an alkoxy group, an halogenoalkyl group, a hydroxyalkyl group, an aminoalkyl group, an alkoxycarbonyl group, a halogen atom, or an amino group; and the remaining one represents a biotin residue or a biotin-crosslinking group).

Herein, a preferred biotin-crosslinking group is represented by the following formula (2):

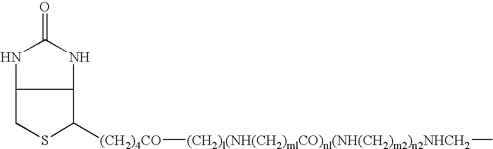

(wherein 1 is a number of 0 to 4, each of m¹ and m² is a number of 1 to 8, and each of n¹ and n² is a number of 0 or 1). The carbon number of the alkyl or alkoxy moiety contained in the above-described alkyl group, alkoxy group, halogenoalkyl group, hydroxyalkyl group, aminoalkyl group, or alkoxycarbonyl group is preferably 1 to 4, particularly preferably 1. Among entities other than the above-mentioned biotin residue or biotin-crosslinking group in R¹ to R⁶, particularly preferred are a hydrogen atom, a methyl group, and a methoxy group.

Examples of particularly preferred biotinylated psoralens include 4′-biotinamidopentylamidohexylaminomethyl-4,5′,8-trimethyl psoralen. These compounds are commercially available from Ambion.

In order to incorporate biotinylated psoralen into a cell, biotinylated psoralen is added to a cell-containing solution. This addition is preferably performed in the presence of a cell-permeability-promoting agent, so as to enhance transfer efficiency of biotinylated psoralen into cells. Examples of the cell-permeability-promoting agent include digitonin, NP-40, Triton X-100, and Tween-20, with digitonin being particularly preferred. The concentration of the biotinylated psoralen employed herein may vary depending on the number of cells, but is preferably from 0.01 to 100 ng/mL, more preferably from 0.05 to 50 ng/mL. The concentration of digitonin is preferably 0.001 to 0.5%, more preferably 0.01 to 0.05%.

No particular limitation is imposed on the long-wavelength UV rays which are employed for exciting biotinylated psoralen, so long as the wavelength falls within a long wavelength range (320-400 nm) of the ultraviolet region. Preferably, the range is 340 to 380 nm, more preferably in the vicinity of 365 nm.

Examples of avidins which have been labeled with a color-developing substance, a fluorescent substance, or a chemiluminescent substance and which are caused to react with biotinylated psoralen that has intercalated into negatively supercoiled DNA when irradiated with UV light include streptavidin and avidin, with streptavidin being preferred.

The color-developing substance serving as a label may be an enzyme label. Examples of the enzyme include peroxidase and alkaline phosphatase. When adivin that has been labeled with any of these enzymes is used, it may be used in combination with a coupler such as 3,3′-diaminobenzidine (DAB), 5-bromo-4-chloro-3-indoryl phosphate (BCIP), or 3,3′-(3,3′-dimethoxy-4,4′-biphenylene)bis[2-(4-nitrophenyl)-5-phenyl-2H-tetrazolium chloride] (NBT), whereby color can be developed. When any of these color-developing substances is used for labeling, sites where crosslink has been induced by psoralen can be detected as pigment deposits of brown or purple color on a chromosome.

The fluorescent substance serving as a label may be fluorescein isothiocyanate; Alexa Fluor 350, 430, 488, 532, 546, 568, 594, 633, 660, or 688; Cy2, Cy3, Cy5; or rhodamine. Use of avidin that has been labeled with any of these fluorescent substances is particularly preferred, as it enables observation under a fluorescence microscope and images can be easily formed.

Examples of the chemiluminescent substance serving as a label include luminol (5-amino-2,3-dihydro-1,4-phthalazinedione), lucigenin(bis-N-methylacridinium nitrate), acridinium esters, adamantly 1,2-dioxetane allyl phosphate, nitric oxides, and bis(2,4,6-trichlorophenyl)oxalate. When any of these chemiluminescent substances is used for labeling, signals can be directly recorded on X-ray film.

As described above, when developed color, emitted fluorescence, or emitted chemiluminescence is measured, it is possible to identify the location of negative supercoiled DNA within the genome DNA. Also, it is possible to detect cells that contain negative supercoiled DNA.

### EXAMPLES

The present invention will next be described in more detail by way of examples, which should not be construed as limiting the invention thereto.

A. Materials and methods

(1) Reagent

Biotinylated psoralen was purchased from Ambion (Brightstar™ Psoralen-Biotin, a compound represented by formula (1), wherein R¹═R³═H, R²═R⁵═R═CH₃, R⁴═biotin-NH(CH₂)₅CONH—(CH₂)₆NHCH₂—). Digitonin was purchased from Calbiochem. α-Amanitin, Br-UTP (bromouridine 5′-triphosphate), and 4,5′,8-trimethylpsoralen were purchased from Sigma. Restriction endonucleases were purchased from Takara Shuzo.

(1) Heat Shock and X-Ray Irradiation

Drosophila melanogaster Oregon R was grown at 18° C. According to the test protocol, some third-instar larvae were subjected to heat shock treatment; i.e., the larvae were collected in a polypropylene tube, and the tube was submerged in a 37° C. water bath for 10 minutes. Salivary glands were obtained through dissection of the larvae in a dissection buffer (10 mM HEPES-KOH (pH 7.6), 5 mM MgCl₂, 5 mM KCl, 130 mM NaCl, 1% polyethylene glycol 6000). Also according to the test protocol, some of the salivary gland specimens were exposed to X-ray irradiation for 60 minutes at a dose rate of about 3 Gy/min (TORREX CABINET X-RAY SYSTEM Model TRX2800, Faxitron), whereby nicks were introduced into DNA.

(2) Staining of Salivary Gland Chromosomes

Four or five pairs of salivary glands were treated with a dissection buffer (40 μL) containing 0.01% digitonin and rinsed with a dissection buffer containing no digitonin. Subsequently, the resultant salivary glands were treated with a dissection buffer containing 0.2 ng/ml biotinylated psoralen for 10 minutes. Thereafter, the salivary glands were irradiated with light from a long-wave (365 nm) UV lamp (UVP model UVL-21), to thereby cause crosslinking of biotinylated psoralen. Upon digitonin treatment, 2 mM Br-UTP was added in the dissection buffer for labeling nascent mRNA. Upon digitonin treatment, 3 μg/mL α-amanitin was added to the dissection buffer, to thereby inhibit transcription by RNA polymerase II. After completion of crosslinking, the salivary glands were fixed with 40% acetic acid, and chromosomes of the salivary glands were squashed onto a glass slide. Br-UTP was detected with anti-BrdU monoclonal antibody (Roche) and rhodamin-labeled anti-mouse IgG antibody.

Biotinylated psoralen was detected with Alexa 488-labeled streptavidin (Molecular Probe). DNA was stained with DAPI. Fluoroimages were analyzed with a Carl Zeiss Axioplan 2 microscope and IP lab software.

(3) Southern Analysis of Crosslinked DNA

Application of heat shock and dissection of larvae were performed in a manner similar to those employed for staining. Twenty pairs of salivary glands were left to stand in a dissection buffer containing psoralen for 10 minutes, and the salivary glands were exposed to 365 nm light for crosslinking. The thus-crosslinked DNA was treated with proteinase K in a lysis buffer (10 mM Tris-Cl (pH 8.2), 100 mM EDTA, 0.5% SDS) for four hours at 55° C., followed by extraction with phenol/chloroform and then with chloroform for purification. The thus-purified DNA was treated with restriction enzymes, and the produced DNA fragments were subjected to a purification procedure. The purified DNA fragments were dissolved in a glyoxal denaturation buffer (1M glyoxal, 10 mM sodium phosphate (pH 7.0), 50% dimethylsulphoxide), and the solution was heated at 50° C. for one hour for denaturation of the DNA fragments. The DNA fragments denaturated with glyoxal were subjected to electrophoresis on 1% agarose gel in 10 mM sodium phosphate buffer (pH 7.0), and a fraction containing DNA fragments to which psoralen had been crosslinked and a fraction containing DNA fragment to which psoralen had not been crosslinked were separated. Electrophoresis was performed at 3.6 volt/cm for 100 minutes.

After electrophoresis, the gel was incubated with a denaturing solution (0.5M NaOH, 1.5M NaCl) at 65° C. for 100 minutes for reverse crosslinking of DNA with psoralen. The DNA fragments in the gel were transferred to a nylon membrane (Hybond-N, Amersham). The membrane was hybridized with ³²P-labeled DNA probe produced through random priming. Hybridization signals were detected by use of X-ray film. The hsp70 CR and DDS regions were excised from a plasmid p56H8RIA (26) as probe templates.

B. Results

(1) Jupe et al. have developed a protocol for quantification of psoralen crosslinking in specific regions in the Drosophila genome using Schneider cells (Non-Patent Document 4). In order to detect negative supercoiled DNA in salivary gland chromosomes, we first attempted to confirm whether the protocol of Jupe et al. can be applied to tissue of, for example, the salivary glands.

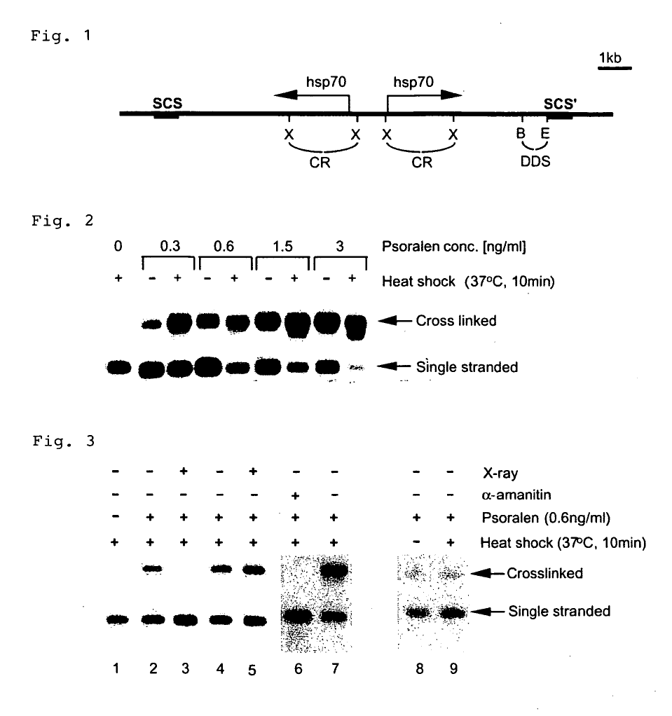

The protocol will be described briefly as follows. The salivary glands were obtained from heat shocked or non-heat shocked larvae. The salivary glands were soaked in buffers containing different concentrations of psoralen, followed by exposure to 365 nm light for crosslinking. DNA contained in each buffer was purified, and the purified DNA was treated with restriction enzymes. The produced DNA fragments were denatured with glyoxal. A fraction containing crosslinked DNA and a fraction containing non-crosslinked DNA were separated from each other through gel electrophoresis, and the gel was treated with an alkali at 65° C. The DNA fragments were blotted onto a nylon membrane. hsp70 located at 87A7 (FIG. 1, CRs and DDS) was analyzed using probes through the Southern method. Incubation of the gel with an alkali at high temperature is indispensable to detection of crosslinked DNA, for the treatment is effective for prevention of self-annealing of the crosslinked DNA in the gel, making signal detection reproducible. We confirmed concentration-dependence of crosslinking occurring in the hsp70-coding region (FIG. 2). The frequency of crosslinking increased when heat shock was applied to the larvae (FIG. 2). When the salivary glands isolated from heat shocked larvae were irradiated with an X-ray to thereby introduce nicks into the DNA and treated with psoralen to thereby cause crosslinking, DNA fragments with substantially no crosslink were observed (FIG. 3 (lanes 3 and 4)). As a control, X-ray irradiation was performed after crosslinking with psoralen, but the results remained unchanged (FIG. 3 (lanes 5 and 2)). This dose of X-rays is estimated to induce about one nick per 30 kb of DNA, suggesting that the size of the relaxed region is greater than the size of the CR fragment (2 kb). Similar results were obtained with the salivary gland chromosomes isolated from non-heat shocked larvae (data not shown). In contrast, the level of crosslinking was low in the hsp70 distal downstream region (DDS) and was not enhanced upon heat shock of larvae (FIG. 3 (lanes 8 and 9)).

These results are in good agreement with those obtained using Schneider cells (Non-Patent Document 4) and indicated that negative supercoils are present in the hsp70-coding region, and that their levels can be increased when heat shock is applied. Therefore, we conclude that the DNA analysis protocol using the psoralen crosslinking is applicable not only to cultured cells but also to the salivary gland tissue.

Interestingly, even in the case where heat-shocked salivary gland chromosomes were subjected to a crosslinking procedure, when the salivary gland chromosomes were treated with α-amanitin prior to the crosslinking step, substantially no crosslink-related signals were detected (FIG. 3, lanes 6 and 7). These observations reveal that negative supercoils are readily relaxed upon inhibition of transcription by RNA polymerase II.

(2) Visualization of Negative Supercoils of DNA on Salivary Gland Chromosomes

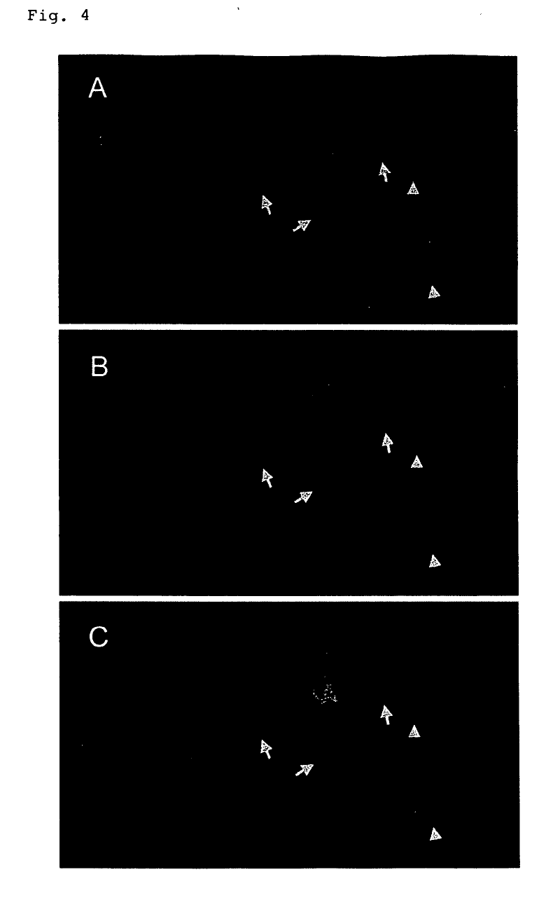

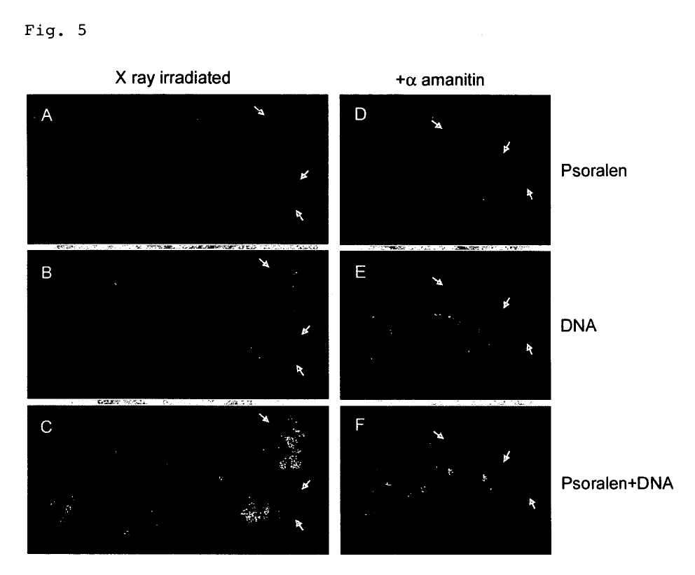

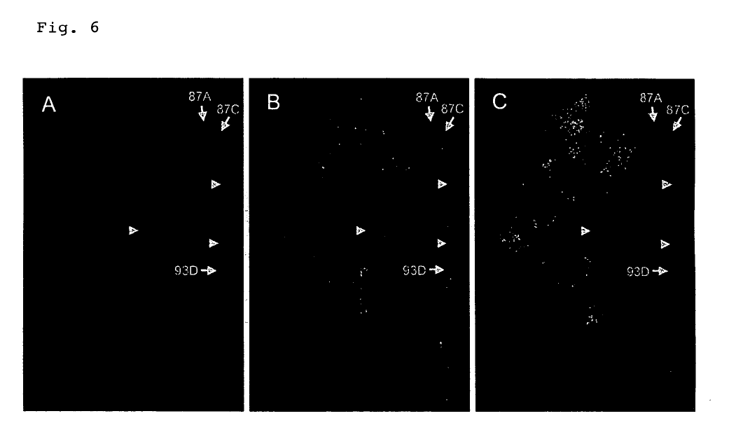

In order to visualize negative supercoils of DNA on salivary gland chromosomes throughout the entire genome, we have extended the psoralen crosslinking protocol, using a protocol in which biotinylated psoralen is detected with fluorescence-labeled streptavidin. Specifically, salivary glands were treated with 0.01% digitonin. Digitonin has been employed to promote permeability of the cell membrane of salivary gland cells and thus is a substance suitably employed in transcription research using salivary gland chromosomes. The salivary glands treated with digitonin were soaked in a buffer containing biotinylated psoralen and exposed to 365 nm light for inducing crosslinking. The resultant salivary gland cells were fixed with acetic acid, and the chromosomes were squashed onto a glass slide. Biotinylated psoralen was detected with Alexa 488-labeled streptavidin. Many bands of psoralen signals were observed in the salivary gland chromosomes (FIG. 4A). There were approximately 150 signals within the entire genome. Such signals were not detected when the chromosomes had been treated with a psoralen-free buffer, or when the crbsslinking step was not performed after treatment with psoralen. Comparison of the thus-obtained signals with the DAPI image of the chromosomes revealed that the regions in which signals from psoralen fell were substantially in agreement with interbands and puffs where fluorescent signals of DAPI were weak (e.g., arrows in FIGS. 4B and 4C), while the vague psoralen signals almost coincided with the DAPI signals (FIGS. 4B and 4C). In some puffs, psoralen signals were observed only on both sides of the puffs (e.g., arrowheads in FIGS. 4B and 4C). When chromosomes had been irradiated with X-ray (180 Gy) before crosslinking with biotinylated psoralen to thereby introduce nicks into the DNA, psoralen signals which may otherwise appear in interbands and puffs were not detected (FIGS. 5A to 5C). In addition, when chromosomes had been treated with α-amanitin, such psoralen signals were also undetected (FIGS. 5D and 5E). As controls, chromosomes which had been similarly treated but had not been undergone X-ray irradiation or α-amanitin treatment exhibited similar patterns as in FIG. 4. These data indicate that, in the interphase genome, transcription-coupled negative supercoils are present in many local domains of the genome. The vague psoralen signals observed in the chromosomes are considered to be attributed to non-specific crosslinking of psoralen to DNA, without any relation to negative supercoils.

(3) Negative Supercoils of DNA Built Up in Heat Shock Puffs

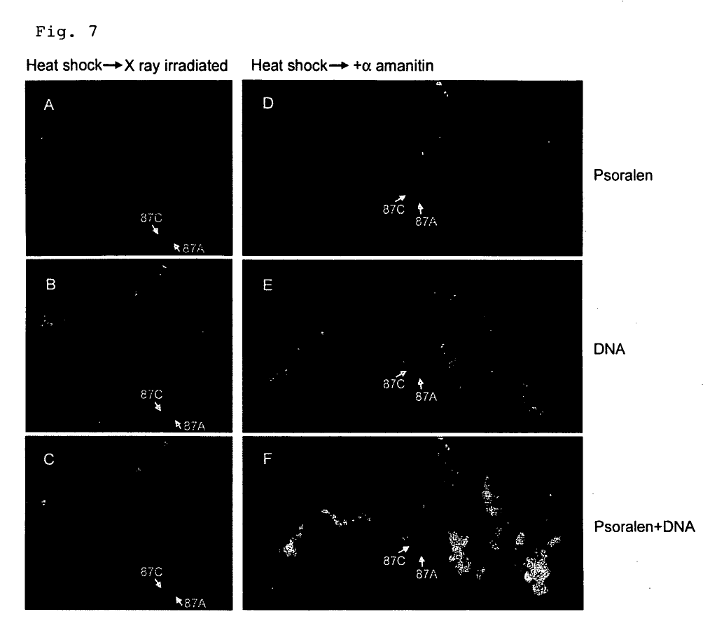

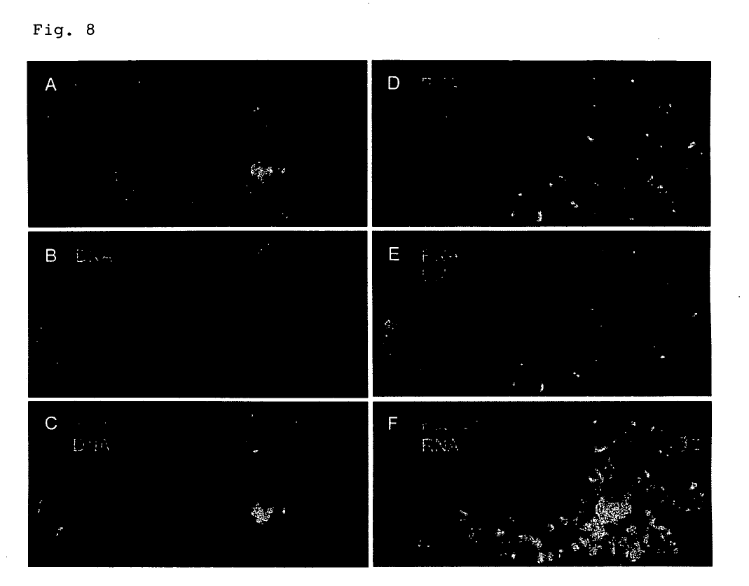

In order to analyze negative supercoils built up in heat shock puffs, salivary glands obtained through dissection of heat shocked larvae were subjected to crosslinking with biotinylated psoralen. In a heat shock puff at 87C, an intensive signal of psoralen was observed (FIG. 6, arrow 87C). In a heat shock puff at 87A, psoralen signals were detected on both sides of the puff but undetected in the central region of the heat shock puff (FIG. 6, arrow 87A). The psoralen signal was also observed on another heat shock puff at 93D (FIG. 6, arrow 93D). Although many psoralen signals were detected in interbands of chromosomes from non-heat shocked larvae, corresponding signals were substantially undetected in the salivary gland chromosomes from heat shocked larvae. X-ray irradiation. (FIGS. 7A to 7C) or α-amanitin treatment (FIGS. 7D to 7F) of salivary glands performed prior to crosslinking did not affect the configuration of heat shock puffs (FIG. 7, arrows), but psoralen signals in heat shock puffs were not detected.

Through labeling of nascent mRNAs with Br-UTP performed after X-ray irradiation, X-ray irradiation was confirmed to have exerted substantially no effect of inhibiting transcription. This suggests that disappearing of psoralen signals after X-ray irradiation is not attributed to the secondary effect of transcription inhibition by X-ray irradiation. Similar results as shown in FIG. 6 were obtained from samples which had undergone a mock treatment with respect to X-ray irradiation or α-amanitin treatment. These data demonstrate that transcription-coupled negative supercoils are present in heat shock puffs.

(4) Correlation between Negative Supercoils and Transcription

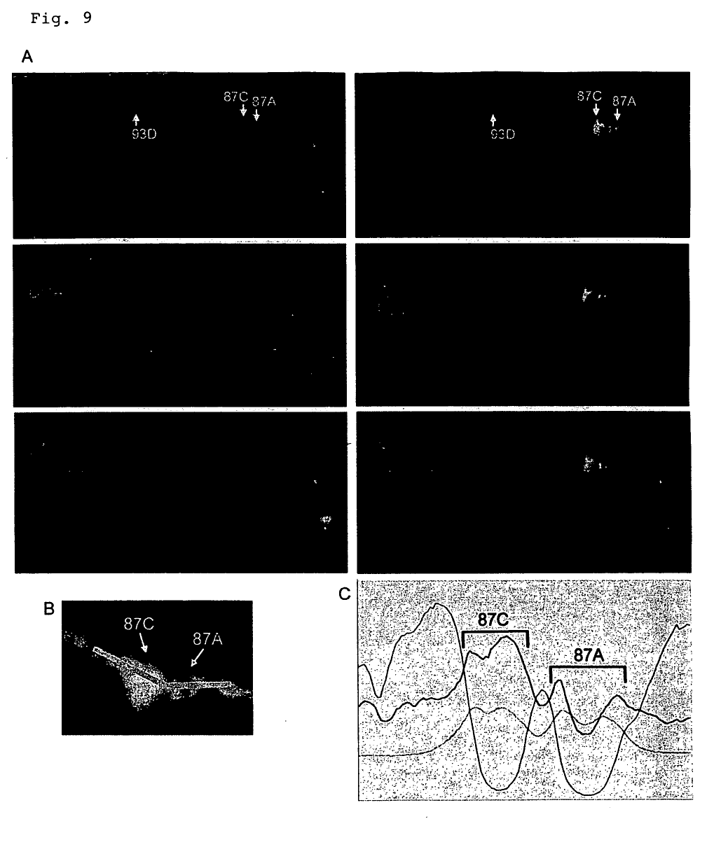

Judging from sensitivity to α-amamtin, generation of negative supercoils in interbands and puffs appears to be coupled with transcription. In order to confirm this, just before crosslinking, salivary glands obtained through dissection were incubated with Br-UTP, to thereby label nascent transcripts. The labeled RNA was detected in many interbands and puffs (FIGS. 8D and 8E). On a merged image, all psoralen signals were highlighted in interbands and puffs where nascent RNAs were present (FIG. 8F).

However, in some interbands or puffs, psoralen signals were not so intensive, although nascent transcripts were clearly detected (FIG. 8F, arrows). After heat shock, remarkable signals attributed to uptake of Br-UTP were observed in the heat shock puffs at 87A and 87C. A weak signal was also observed in the heat shock puff at 93D (FIG. 9). These data coincide with the previous report (Nature, 370: 75-77 (1994)) stating that the C-terminal domain phosphorylated RNA polymerase II localizes exclusively in heat shock puffs. A strong psoralen signal was detected in the heat shock puff at 87C, but no psoralen signal was detected in the central region of the heat shock puff at 87A. For detailed investigation on distribution of negative supercoils and transcripts, signal intensities of psoralen, RNA, and DNA were quantified in the 87A and 87C puffs and surrounding portions thereof (FIGS. 9B and 9C). The distribution of signals of psoralen and RNA was found not to be uniform within the 87A and 87C puffs, but signal patterns were very similar to each other. The intensity of DNA signals was extremely low in these puffs. Summing up the quantification data, close correlation between negatively supercoiled DNA and transcription has been established. Negatively supercoiled DNA is considered to be in an equilibrium state in terms of their generation and relaxation. The fact that the ratio of signal intensity of psoralen to that of nascent RNA differs depending on the identities of interbands and puffs suggests that the equilibrium state also differs from region to region within the genome.

Through a conventional method employing the Southern method, negative supercoils had been detected in only two regions of the entire genome (Non-Patent Document 4). Therefore, we conjectured that psoralen signals would be detected in only several regions of the genome. However, quite unexpectedly, many psoralen signals were observed in the salivary gland chromosomes. Such signals were detected in many interbands or puffs in which transcription was activated, but not detected in every interband or puff. When nicks had been introduced into DNA before crosslinking, or transcription had been inhibited before crosslinking, pshoralen signals were not detected. Thus, the present invention is the first to visualize negatively supercoiled DNA on interphase chromosomes. Various effects of the present invention are realized since genome can be directly stained according to the present invention.

Effects of the Invention

According to the present invention, negative supercoils of DNA, which have conventionally been detected through a considerably complex procedure only in limited portions of genome, can be detected conveniently and efficiently.

1. A method for detecting negatively supercoiled DNA in cells, characterized by including the steps of incorporating biotinylated psoralen into cells, irradiating the cells with long-wavelength UV rays, causing the cells to react with adivin which has been labeled with a color-developing substance, a fluorescent substance, or a chemiluminescent substance, and measuring developed color, emitted fluorescence, or emitted chemiluminescence of the cells.

2. A method for detecting a cell containing negatively supercoiled DNA, characterized by including the steps of incorporating biotinylated psoralen into cells, irradiating the cells with long-wavelength UV rays, causing the cells to react with adivin which has been labeled with a color-developing substance, a fluorescent substance, or a chemiluminescent substance, and measuring developed color, emitted fluorescence, or emitted chemiluminescence of the cells.

3. The detection method according to claim 1 or 2, wherein the cells are eukaryotic cells.

4. The detection method according to any one of claims 1 to 3, wherein incorporation of biotinylated psoralen into cells is performed in the presence of a cell membrane permeation promoting agent.
